(19) United States
(12) Patent Application Publication
(10) Pub. No.: US 2009/0306402 A1
(43) Pub. Date: Dec. 10, 2009

(54) Composition of N-Alkenyl Carboxylic Acid Tertiary Amide

(75) Inventors: Shukichi Ugamura, Osaka (JP); Kenichi Takematsu, Tokyo (JP)

Correspondence Address: BUCHANAN, INGERSOLL & ROONEY PC, POST OFFICE BOX 1404, ALEXANDRIA, VA, 22313-1404, US

(73) Assignee: NIPPON SHOKUBAI CO., LTD., Osaka-shi (JP)

(21) Appl. No.: 11/919,340

(22) PCT Filed: Apr. 27, 2006
(86) PCT No.: PCT/JP2006/309214

§ 371 Date: Oct. 26, 2007

(30) Foreign Application Priority Data
Apr. 28, 2005 (JP) 2005-130641

(51) Int. Cl. C07D 207/12

(52) U.S. Cl. 548/401

(57) ABSTRACT

A composition of N-alkenyl carboxylic acid tertiary amide which enables to suppress variation of pH and color valency and realize storage stability for a long period, by the addition of specified amines.

### TECHNICAL FIELD

The present invention relates to a composition of N-alkenyl carboxylic acid tertiary amide, having stability for a long period, by the addition of specified amines.

### BACKGROUND ART

A composition of N-alkenyl carboxylic acid tertiary amide has a property to apt to polymerize during keeping, transportation, storage, and the like.

Therefore, the addition of ammonia, N,N′-sec-butyl-p-phenylenediamine, sodium hydroxide, and the like has been proposed aiming at suppression of polymerization (JP-A-8-506580).

In addition, there is such description that other amines with N,N′-sec-butyl-p-phenylenediamine may be used to prevent polymerization, however, there is no description or suggestion on what kind of amines are suitable for stability of color valency (APHA) (JP-A-7-252221).

### DISCLOSURE OF INVENTION

N,N′-sec-butyl-p-phenylenediamine (an aromatic amine having phenylenediamine skeleton) described in JP-A-8-506580 exhibits only low effect as a stabilizer when used in small amount, therefore the addition thereof in large quantity is required to express the effect. However, our study clarified that the addition of the compound in large quantity causes coloring of a product.

In addition, in JP-A-7-252221, N,N′-sec-butyl-p-phenylenediamine or the like was added to prevent polymerization in a formation step and not to maintain pH or reduce color valency (APHA) aiming at stability. Namely, it is not aimed at storage stability in the present invention, and thus it is hard to say that a problem of storage stability is solved.

N-alkenyl carboxylic acid tertiary amide has conventionally been well-known to be labile to radical polymerization, and thus paying close attention is required during keeping, transportation and storage. Accordingly, it is an object of the present invention to make possible stable storage of N-alkenyl carboxylic acid tertiary amide for a long period.

The present invention relates to a composition of N-alkenyl carboxylic acid tertiary amide, characterized by containing N-alkenyl carboxylic acid tertiary amide and an amine compound (A) not having phenylenediamine skeleton.

According to the present invention, a composition of N-alkenyl carboxylic acid tertiary amide having small pH variation, and small increase in color valency (APHA) and superior storage stability for a long period, can be provided, by compounding specified amines and limiting pH range, if necessary.

### BEST MODE FOR CARRYING OUT THE INVENTION

We have intensively studied on stable storage of N-alkenyl carboxylic acid tertiary amides for a long period, and have found that variation of pH or color valency (APHA) can be suppressed and stable storage is possible for a long period at a temperature range of 30° C., by the addition of specified amines to N-alkenyl carboxylic acid tertiary amides, and adjusting pH thereof in a range of 8 to 12, if necessary. In this connection, use of “preservation” instead of “storage” is also within the scope of the present invention. The present invention also includes not only simple storage but also transfer or transportation.

As N-alkenyl carboxylic acid tertiary amides used in the present invention, N-linear alkenyl carboxylic acid amides such as N-vinyl formamide; N-alkenyl cyclic carboxylic acid amides such as N-vinyl-ε-caprolactam or N-vinylpyrrolidone (NVP); and the like are included.

Content of N-alkenyl carboxylic acid tertiary amides in a composition of the N-alkenyl carboxylic acid tertiary amide is not especially limited as long as being not lower than 80% by weight in total composition, however, preferably not lower than 85% by weight, more preferably not lower than 90% by weight and most preferably not lower than 95% by weight. The content within this range enables to sufficiently express storage stability of the composition.

A buffer agent or a deodorant, or water or alcohols as a solvent may be added into the present composition in addition to N-alkenyl carboxylic acid tertiary amides, within a range not to lower storage stability over a long period.

Amines to be added in the present invention include any amine as long as not having phenylenediamine skeleton. Phenylenediamine skeleton here means one represented by chemical formula 1:

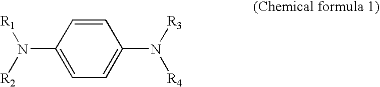

wherein R¹ to R⁴ each independently represent alkyl groups having carbon atoms of not more than 15.

As an amine compound (A) not having phenylenediamine skeleton, an organic amine compound is preferable in view of excellent stabilization effect, and furthermore, an organic amine compound having a vapor pressure at 20° C. of not lower than 3×10⁻⁶ hPa is preferable. Specific examples thereof include aliphatic amines, alkanol amines, hydroxylamines, and the like.

Specifically, the following amines are preferable. (An Amine Compound (A) not Having Phenylenediamine Skeleton)

Aliphatic amines such as methylamine (MA), ethylamine (EA), isopropylamine, t-butylamine (tBA), dimethylamine, diethylamine, triethylamine, and cyclohexylamine;

Alkanol amines such as monoethanol amine (MEA), diethanol amine, and triethanol amine;

Hydroxylamines such as N,N-dimethylhydroxylamine; ammonia;

Aromatic amines such as aniline, toluidine, benzylamine, 2-naphthylamine, and p-aminobenzoic acid;

Heterocyclic aromatic amines such as 4-aminoquinoline.

Aromatic amines (B) which can be used in combination with the amine compound (A), in particular, aliphatic amines, alkanol amines, and hydroxylamines, any amines as long as having an aromatic ring are included, specifically aromatic amines such as aniline, toluidine, benzylamine, 2-naphthylamine, p-aminobenzoic acid, and N,N′-di-sec-butyl p-phenylenediamine (KEROBIT trade mark of BASF AG); heterocyclic aromatic amines such as 4-aminoquinoline; and the like are included.

The amine compound (A) may be used singly or as in a mixed form of two or more kinds. Furthermore, it may be used in combination with an aromatic amine (B), and in this case, two or more kinds of aromatic amines (B) may be added.

Content of the amine compound (A) based on the total composition depends on the kind of the amine to be added, and can be determined within a range enabling to efficiently express storage stability for a long period.

The addition amount of the amine compound (A) may be controlled in compounding so that pH determined by a specified pH measurement method, after storage at 30° C. for 3 months under air atmosphere is in a range of 8 to 12, and preferably in a range of not lower than 1 ppm and not higher than 3% by weight is enough, and preferably in a range of not lower than 1 ppm and not higher than 1% by weight, more preferably in a range of not lower than 3 ppm and not higher than 5000 ppm, and most preferably in a range of not lower than 5 ppm and not higher than 1000 ppm, based on N-alkenyl carboxylic acid tertiary amides. By controlling the amount within this range, the effect of excellent storage stability of the composition for a long period can sufficiently be fulfilled.

On the other hand, ratio of an aromatic amine (B) to the amine compound (A) depends on a kind of an amine to be added, however, can be determined among the combinations with the amine compound (A) to afford to efficiently express storage stability. Specifically, the aromatic amine (B) is not higher than ½, preferably not higher than ⅕ and most preferably not higher than 1/10 of the amine compound (A), in weight ratio. It is because this range enables to express stabilization effect of the composition. The addition amount satisfying the above condition and in a range of, usually not lower than 1 ppm and not higher than 1% by weight is enough, and preferably it is in a range of not lower than 1 ppm and not higher than 5000 ppm, more preferably in a range of not lower than 3 ppm and not higher than 1000 ppm, and most preferably in a range of not lower than 3 ppm and not higher than 500 ppm, based on N-alkenyl carboxylic acid tertiary amides.

To make the composition furthermore stable, pH is controlled preferably in a range of 8 to 11, more preferably 8.5 to 10.5.

A composition of the present invention, in some cases, keeps stability at a high temperature of 90° C. over 2 months, and thus can be said as a composition with very high stability.

Means to produce a composition of the present invention includes just the addition of specified amines to a composition of highly pure N-alkenyl carboxylic acid tertiary amide obtained via a purification step such as distillation, or to a composition of N-alkenyl carboxylic acid tertiary amide obtained without purification. The addition order of the amine compound (A) and the aromatic amine (B) is not especially limited, and the simultaneous addition is also possible.

For storage of a composition of N-alkenyl carboxylic acid tertiary amide, a sealed tank, drum, and the like can be used.

Atmosphere of the vapor phase part of a storage container of a composition of N-alkenyl carboxylic acid tertiary amide used in the present invention is not especially limited, however, handling by avoiding contact with acidic gas such as carbon dioxide gas is preferable, specifically, inert gas such as nitrogen gas is preferable. A composition of N-alkenyl carboxylic acid tertiary amide may be charged in a container after filling with inert gas, in advance, or inert gas may be filled after a composition of N-alkenyl carboxylic acid tertiary amide is charged.

### EXAMPLES

The present invention is explained more specifically by referring to Examples below. However, the following Examples are only for explanation purpose and should not limit the present invention.

An acceleration test method and a measurement method for pH and color valency (APHA) to confirm stability are shown below.

### (Test Methods)

A composition sample of 100 g is charged in a 100 mL screw tube, which is capped and stored in an oven (produced from Sanyo Electric Co., Ltd.) set at 30° C. under air atmosphere.

Property values are measured by the following methods, before the storage and after three months have passed.

### (Measurement Method for pH)

Deionized water of 45 g which is adjusted to have a pH of 6 to 7 is weighed into a 50 mL screwed tube, in which 5 g of the sample is then weighed and mixed to produce a 10% by weight of an aqueous solution of the composition. A pH meter is set within 1 minute, and after stirring at 200 rpm, at 25° C. for 1 minute, it is subjected to standing still and pH value at this point is read.

A pH meter (F-12 model; electrode type #6366-10D, produced from Horiba, Ltd.)

### (Measurement Method for APHA Color Valency)

A color test reagent (Color 1000: produced from Wako Pure Chemical Industries, Ltd.) is diluted with deionized water to prepare various color standard solutions. A composition sample of 25 mL is charged in a 25 mL calorimetric tube, and APHA No, showing the same color, compared with standard solutions, is determined as measurement value.

### Examples 1 to 12 and Comparative Examples 1 to 4

NVP is added with various amines, and pH and color valency (APHA) thereof are measured at the start of storage and after 3 months have passed, whose results are shown in the following Table 1.

In this connection, NVP used in Examples 1 to 14 and Comparative Examples 1 to 6 are commercial NVP (containing 10 ppm of a stabilizer: N,N′-sec-butyl-p-phenylenediamine) after purification treatment such as distillation, and after removal of the stabilizer. As an example of such purification treatment, refer to Japanese patent No. 3435598.

TABLE 1

| | | Amines | | | | After 3 months | |
|---|---|---|---|---|---|---|---|
| | | | Aromatic amine | Initial value | | | Color |
| | Composition | Amine comp. (A) | comp. (B) | | Color valency | | valency |
| | (%) | (ppm) | (ppm) | pH | (APHA) | pH | (APHA) |
| Exp. 1 | NVP (100) | MEA (100) | — | 10.3 | ≦5 | 9.8 | 35 |
| Exp. 2 | ↑ | MA (500) | — | 10.7 | ≦5 | 10.0 | 35 |
| Exp. 3 | ↑ | EA (500) | — | 10.3 | ≦5 | 9.7 | 35 |
| Exp. 4 | ↑ | tBA (10) | — | 10.2 | ≦5 | 9.5 | 40 |
| Exp. 5 | ↑ | MEA (50) | NN (10) | 10.3 | ≦5 | 9.6 | 40 |
| Exp. 6 | ↑ | MA (300) | NN (10) | 10.5 | ≦5 | 10.0 | 30 |
| Exp. 7 | ↑ | EA (1000) | NN (100) | 11.1 | ≦5 | 10.5 | 45 |
| Exp. 8 | ↑ | tBA (100) | NN (10) | 10.4 | ≦5 | 9.9 | 35 |
| Exp. 9 | NVP (90)/ | MEA (100) | — | 10.3 | ≦5 | 9.5 | 40 |
| | Deionized | | | | | | |
| | water | | | | | | |
| | (10) | | | | | | |
| Exp. 10 | ↑ | MA (100) | — | 10.3 | ≦5 | 9.1 | 45 |
| Exp. 11 | NVP (100) | MA (50) | — | 10.4 | ≦5 | 9.2 | 40 |
| | | MEA (50) | | | | | |
| Exp. 12 | ↑ | MA (50) | NN (10) | 10.5 | ≦5 | 9.2 | 40 |
| | | MEA (50) | | | | | |
| Comp. | ↑ | — | — | 9.7 | ≦5 | 6.3 | 200 |
| Exp. 1 | | | | | | | |
| Comp. | ↑ | — | NN (100) | 10.4 | ≦5 | 6.9 | 200 |
| Exp. 2 | | | | | | | |
| Comp. | ↑ | — | NN (500) | 10.7 | ≦5 | 7.1 | 200 |
| Exp. 3 | | | | | | | |
| Comp. | ↑ | — | NN (5 wt %) | 12.5 | 40 | 9.6 | 350 |
| Exp. 4 | | | | | | | |

In this Table, “NN” represents N,N′-sec-butyl-p-phenylenediamine.

As is shown in Examples 1 to 12, by the addition of the amine compound (A) and the aromatic amine (B), if necessary, pH value little lowered compared with the value before starting the storage, and furthermore, variation range of color valency (APHA) could be controlled within 50, and therefore, these specified amines were found to significantly contribute to stability.

On the contrary, in Comparative Examples 1 to 3, pH decreased largely and variation range of color valency (APHA) was also large, due to no addition of the amine compound (A), and stability can be said insufficient.

Further, in Comparative Example 4, variation range of pH, in particular, color valency (APHA) was large, due to the excess addition of an amine having phenylenediamine skeleton, and stability was also insufficient.

From these results of the acceleration test, the present invention is assumed to be excellent even under a usual storage method.

### Example 13 and Comparative Example 5

NVP was charged, in the composition shown in Table 2, in a 200 kg content chemical drum (an inner bag was made of polyethylene). Values of pH and color valency (APHA) thereof were measured at the start of storage and after 3 months have passed, whose results are shown in Table 2. Storage condition was at room temperature (20° C. to 25° C.).

### Example 14 and Comparative Example 6

NVP was charged in the composition shown in Table 2, in an 18 L hybrid drum and nitrogen gas was then filled and the drum was sealed. Values of pH and color valency (APHA) thereof were measured at the start of storage and after 3 months have passed, whose results are shown in Table 2. Storage condition was at room temperature (20° C. to 25° C.).

TABLE 2

| | | Amines | | | | | |
|---|---|---|---|---|---|---|---|
| | | Amine | Aromatic | | | After 3 months | |
| | | comp. | amine | Initial value | | | Color |
| | Composition | (A) | comp. (B) | | Color valency | | valency |
| | (%) | (ppm) | (ppm) | pH | (APHA) | pH | (APHA) |
| Exp. 13 | NVP (100) | MEA (50) | NN (10) | 10.7 | 10 | 10.5 | 50 |
| Exp. 14 | NVP (100) | MEA (50) | NN (10) | 10.7 | 10 | 10.8 | 45 |
| Comp. | NVP (100) | — | — | 8.5 | 5 | 6.4 | 300 |
| Exp. 5 | | | | | | | |
| Comp. | NVP (100) | — | — | 10.8 | 10 | 7.0 | 300 |
| Exp. 6 | | | | | | | |

As is shown in Table 2, a composition of the present invention was confirmed to be excellent even when stored under practical storage condition.

The above contents are preferable embodiments of the present invention, and various changes and modifications should be understood to be within the scope of the present invention without departing from the spirit and scope of the present invention.

1-14. (canceled)

15. A composition of N-vinyl-pyrrolidone, comprising at least one amine selected from the group consisting of an alkanol amine, methylamine, ethylamine, isopropylamine, t-butylamine, dimethylamine, diethylamine, and cyclohexylamine,
wherein content of the amine is in a range of not lower than 1 ppm and not higher than 3% by weight, based on weight of the N-vinyl-pyrrolidone.

16. A composition of claim 15, wherein the amine is an alkanol amine.

17. A composition of claim 16, wherein the alkanol amine is an amine having a vapor pressure at 20° C. of not lower than 3×10⁻⁶ hPa.

18. A composition of claim 16, wherein the alkanol amine is at least one member selected from a group consisting of monoethanol amine, diethanol amine, and triethanol amine.

19. A composition of claim 15, further comprising an aromatic amine (B).

20. A composition of claim 19, wherein the aromatic amine (B) is at least one member selected from a group consisting of aniline, toluidine, benzylamine, 2-naphthylamine, N,N′-di-sec-butyl p-phenylenediamine and p-aminobenzoic acid.

21. A composition of claim 15, further comprising a solvent.

22. A composition of claim 21, wherein content of the solvent is up to 20% by weight based on total weight of the resultant composition.

23. A composition of claim 15, wherein pH determined by a specified pH measurement method, after subjecting the composition to an acceleration test at 30° C. for 3 months under air atmosphere, is in a range of 8 to 12.

24. A composition of claim 15, wherein variation of APHA color valency determined by a specified pH measurement method, after subjecting the composition to an acceleration test at 30° C. for 3 months under air atmosphere, is within 50 compared with the value before storage.
